(19) United States
(12) Patent Application Publication
(10) Pub. No.: US 2012/0078233 A1
(43) Pub. Date: Mar. 29, 2012

(54) WOUND BED PREPARATION

(75) Inventors: Ernest A. Dion, Danvers, MA (US); Ewan Mackenzie, Boston, MA (US); Richard Bevier, Sarasota, FL (US)

(73) Assignee: Smith & Nephew, Inc., Memphis, TN (US)

(21) Appl. No.: 13/315,702

(22) Filed: Dec. 9, 2011

Related U.S. Application Data
(62) Division of application No. 12/521,464, filed on Dec. 22, 2009, now abandoned.
(60) Provisional application No. 60/882,735, filed on Dec. 29, 2006.

(51) Int. Cl. A61B 17/00

(52) U.S. Cl. 604/540

(57) ABSTRACT

A wound bed preparation device for delivering a fluid jet to tissue includes a handpiece having a deck height in the range of −0.0254 mm to 0.2032 mm, a channel width in the range of 0.8636 mm to 1.1684 mm, and a nozzle diameter in the range of 0.10668 mm to 0.12192 mm. The handpiece has a constant channel width over a majority of a tissue impacting section of the handpiece.

### TECHNICAL FIELD

This application relates to wound bed preparation.

### BACKGROUND

Wound bed preparation assists in obtaining maximum benefits from wound care treatment and offers opportunities for the management of chronic wounds. Preparing the wound bed can be achieved by the removal of barriers, such as foreign particles and necrotic or compromised tissue. The presence of necrotic or compromised tissue is common in chronic non-healing wounds, and its removal has many beneficial effects, including removing bacteria and cells that impede the healing process thereby stimulating the build-up of healthy tissue.

Known surgical techniques for wound bed preparation include surgical and sharp debridement (joint is flushed with fluid and damaged tissue is removed with a sharp scalpel, scissors, or similar instrumentation); autolytic debridement (the body removes dead tissue through enzymes present in the wound liquefying non-viable tissue, which can be fostered by physicians utilizing moist wound dressings); biological debridement (larval or maggot therapy); enzymatic debridement (use of preparations known as exogenously derived proteolytic enzymes such as streptokinase or papain-urea preparations to trigger and promote hydrolysis and degradation of the proteinaceous devitalized tissue); and chemical debridement (topical application of relatively caustic chemicals such as calcium or sodium hypochlorite solutions or other chemicals to the wounded area).

### SUMMARY OF THE INVENTION

The wound bed preparation techniques described below enable a surgeon to simultaneously hold, cut, and remove damaged tissue and contaminants while limiting collateral trauma. The techniques enable the surgeon to place a wound bed preparation device directly onto the wound bed with a light touch and remove precise layers of the tissue with each stroke of the device across the wound bed. Surgical debridement is accomplished in a single step and utilizes a relatively small amount of irrigant which is immediately evacuated, minimizing saturation of the operative field and reducing the risk of splashing and aerosolization, which can create hazards within the operating suite.

In one general aspect, a wound bed preparation device for delivering a fluid jet to tissue, comprises a handpiece having a deck height in the range of −0.0254 mm to 0.2032 mm.

Implementations can include one or more of the following features. For example, the device has a channel width in the range of 0.8636 mm to 1.1684 mm. The device has a nozzle diameter in the range of 0.10668 mm to 0.12192 mm.

In another general aspect, a wound bed preparation device for delivering a fluid jet to tissue, comprises a handpiece having a constant channel width over a majority of a tissue impacting section of the handpiece, the constant channel width being in the range of 0.8636 mm to 1.1684 mm.

Implementations can include one or more of the following features. For example, the handpiece includes a distal tip configured to perform a surgical procedure at a surgical site, the distal tip defining a channel, a deck, and a backside, and wherein the channel extends from the backside of the distal tip to the deck, and further comprises a first conduit disposed within the channel and configured to deliver fluid under high pressure to the surgical site, the first conduit comprising a nozzle having a fluid opening and positioned such that a distance between a center of the fluid opening and the deck corresponds to the deck height. The device further comprising a second conduit coupled to the handpiece, the second conduit configured for the removal of fluid and debris from the surgical site. The distance between the center of the fluid opening and the deck is about 0.0127 mm +0.1270 mm −0.0254 mm. The first conduit and the second conduit are connected to the distal tip. The first conduit wraps around a portion of the distal tip at a bend defined between the backside of the distal tip and the deck such that fluid exiting from the first conduit is directed proximally in the vicinity of the deck toward the second conduit. The distal tip defines a second channel in fluid-flow communication with the nozzle and having a channel width in the range of 0.8636 mm to 1.1684 mm. The channel width is about 1.016 mm +/−0.1270 mm. The second channel is U-shaped. The second channel flares in a proximal region of the second channel to limit impingement of the fluid on the second channel as the fluid diverges proximally. The nozzle diameter is about 0.1143 mm +/−0.00762 mm. The device further comprising a console configured to provide high pressure fluid to the handpiece. The device further comprising a tubing coupled between the console and the handpiece. The device further comprising a pump assembly coupled to the console and the handpiece and configured to provide high pressure fluid to the handpiece. The device further comprising a feed conduit configured to be coupled to a fluid source to provide fluid to the pump assembly.

In another general aspect, a surgical method comprises placing a distal tip of a handpiece directly onto a wound bed; and while maintaining contact between the distal tip and the wound bed, delivering fluid under pressure to the wound bed via a nozzle in the vicinity of a deck formed by distal tip to remove layers of tissue from the wound bed, the nozzle defining a fluid opening and positioned such that a distance between a center of the fluid opening and the deck is in the range of −0.0254 mm to 0.2032 mm.

Implementations can include one or more of the following features. For example, delivering fluid to the wound bed further comprises receiving fluid exiting the nozzle through a channel defined by the distal tip and in fluid-flow communication with the nozzle. Delivering fluid under pressure to the wound bed comprises passing the distal tip across the wound bed in a substantial back and forth motion while maintaining contact between the distal tip and the wound bed.

### DESCRIPTION OF DRAWINGS

FIG. 1 is an illustration of the wound bed preparation device.

FIG. 2 is an illustration of the components of a handset of the device.

FIG. 3 is a side view of a tube assembly of a handpiece of the handset.

FIG. 4 is a perspective deckside view of a distal tip of the tube assembly.

FIG. 5 is a perspective backside view of the distal tip.

FIG. 6 is a cross-sectional view of the distal tip.

FIG. 7 is a backside view of the distal tip.

FIG. 8 is a cross-sectional view of a jet tube of the tube assembly.

FIG. 9 is a side view of the distal tip.

FIG. 10 is a deckside view of the distal tip.

FIG. 11 is an end view of the distal tip along lines 11-11 in FIG. 10.

### DETAILED DESCRIPTION

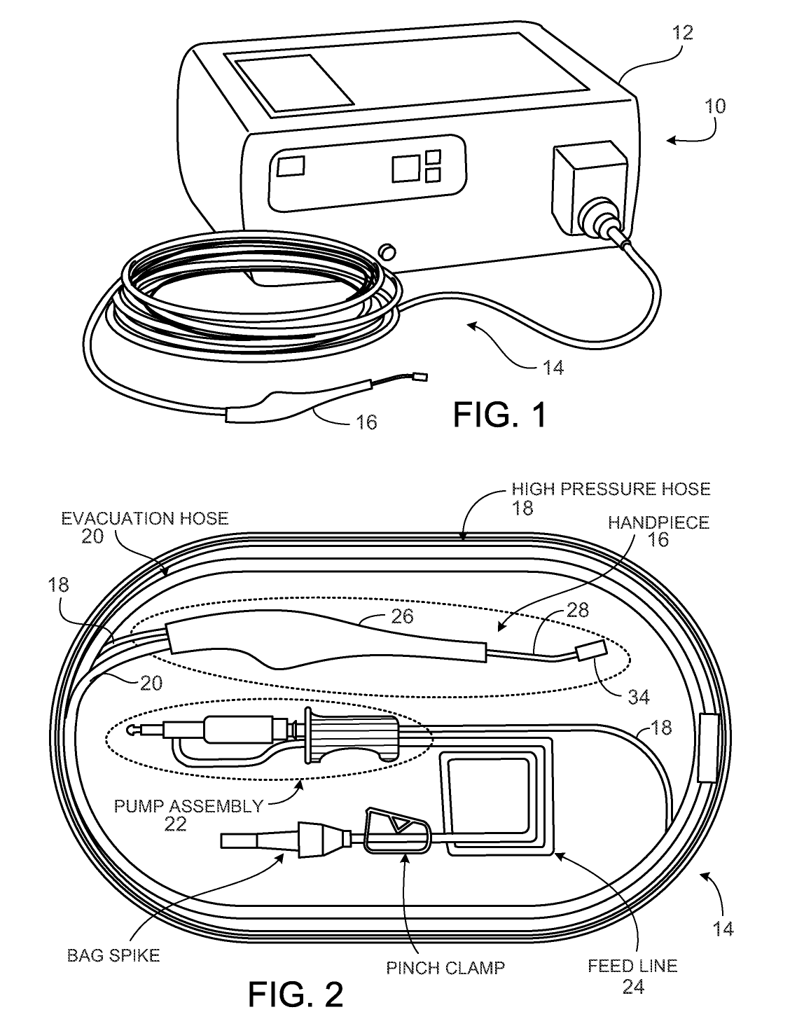

Referring to FIGS. 1 and 2, a wound bed preparation device 10 includes a console 12 and a handset 14. The handset 14 includes a handpiece 16, which a surgeon operates to cut, clean, and debride a wound, a high pressure hose 18 for delivering fluid under high pressure to the handpiece 16, and an evacuation tube 20 for the removal of fluid and debris from the surgical site. The handpiece 16 is connected to a pump assembly 22 by the high pressure hose 18. The pump assembly 22 connects to console 12 to provide high pressure fluid to the handpiece 16 through the high pressure hose 18, as described in U.S. Published application numbers 2003/0125660, 2004/0234380, and 2006/0264808, hereby incorporated by reference in their entirety.

Also connected to the pump assembly 22 is a feed line 24 that can be connected to a source of fluid, for example, a saline bag (not shown). Fluid from the feed line 24 is pressurized in the pump assembly 22 and delivered to the handpiece 16. Handpiece 16 includes a housing 26 and a tube assembly 28 located within and extending from the housing 26. Except for the configuration of the distal tip 34 of the handpiece 16, described below, and the nozzle diameter, described below, the device 10 corresponds to the Versajet™ Hydrosurgery System 1 and Versajet™ Plus, available from Smith & Nephew, Inc., catalog numbers Console #50700 (115V), Console #50750 (230V), Versajet™ 1 Handset #50635 (14 mm/15°), #50636 (14 mm/45°), and #50637 (8 mm/45°), and Versajet™ Plus Handset #52365 (14 mm/15°), #52636 (14 mm/45°), and #52637 (8 mm/45°).

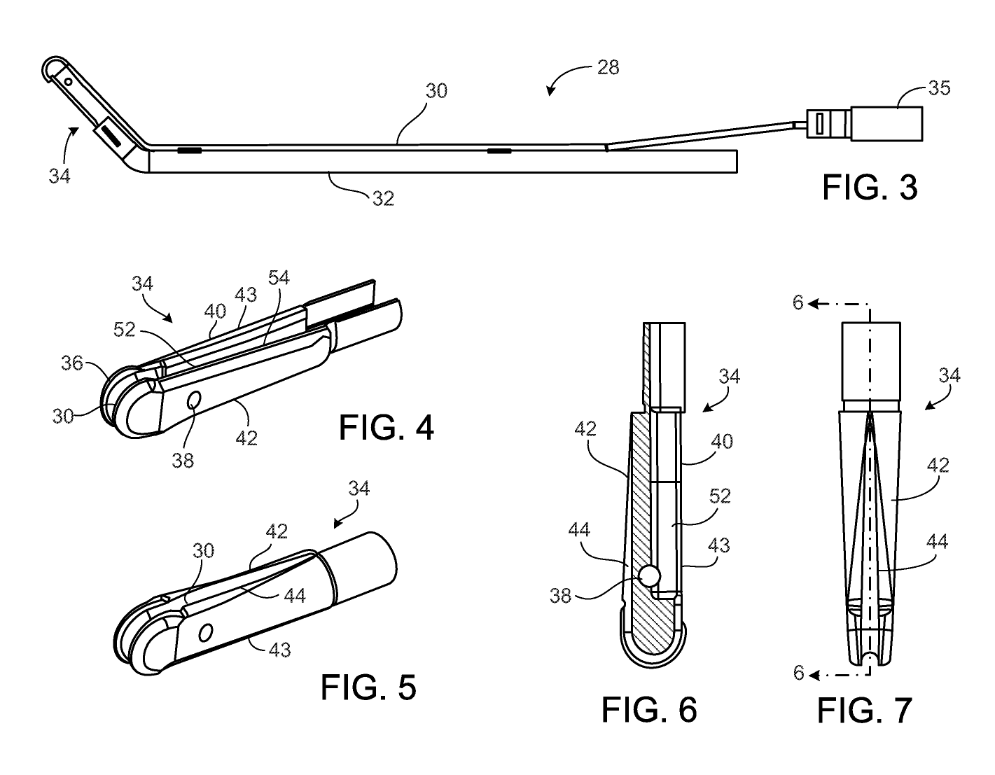

Referring to FIG. 3, tube assembly 28 includes a jet tube 30 that connects to the high pressure hose 18, and an evacuation tube 32 that connects to evacuation tube 20. The jet tube 30 and the evacuation tube 32 are connected to the distal tip 34, and the jet tube 30 is also connected to a filter 35, which is configured to remove any unwanted particles from the high pressure fluid stream. Referring also to FIG. 4, the jet tube 30 wraps around the distal tip 34 at bend 36 from a backside 42 of the distal tip, such that fluid exiting from the jet tube 30 is directed proximally along a deckside 43 of the distal tip 34 toward the evacuation tube 32. The high pressure fluid exiting from the jet tube 30 acts to treat the tissue in the vicinity of a deck 40 of the distal tip 34, and the fluid along with removed tissue is drawn into the evacuation tube 32 by a venturi effect. The distal tip 34 has a vent hole 38 for purposes described in US 2003/0125660, supra.

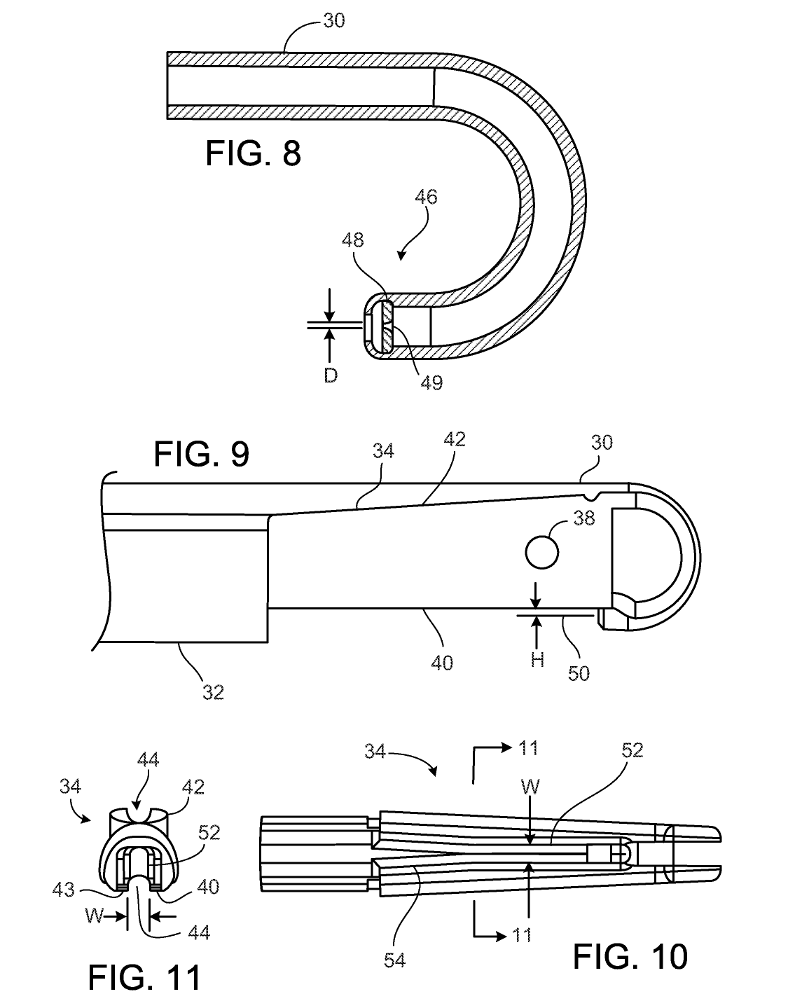

Referring to FIGS. 5-7, the distal tip 34 defines a channel 44 that runs from the backside 42 of the distal tip 34 around the bend 36 and to the deck 40. The jet tube 30 is positioned in the channel 44, as shown in FIGS. 4 and 5. Referring to FIG. 8, at an end 46 of the jet tube 30 is a nozzle 48, as described in US 2006/0264808, supra, having a fluid exit hole 49 from which exits the high pressure fluid for treating tissue. Referring to FIG. 9, the relative position of the center 50 of the fluid exit hole 49 and the deck 40, referred to as the deck height, H, is critical to the use of the device 10 in wound bed preparation. The larger the deck height, H, the more aggressive and less precise the tissue treatment and rougher resultant tissue bed; the smaller the deck height, H, the less aggressive and more precise the tissue treatment and smoother resultant tissue bed.

Referring to FIGS. 10 and 11, the distal tip 34 defines a U-shaped channel 52 along which fluid exiting from the nozzle 48 travels. Due to the venturi effect created by the high pressure fluid entering the evacuation tube 32, suction is applied along the channel 52 to the tissue being treated. The channel width, W, is critical to the use of the device 10 in wound bed preparation. The larger the width, W, the more aggressive and less precise the tissue treatment due to the effect the width has on the amount of tissue drawn into the channel and thus impacted by the fluid jet; the smaller the width, the less tissue drawn into the channel, and the less aggressive more precise the tissue treatment. The channel 52 flares in a proximal region 54 of the channel to limit impingement of the fluid jet on the channel walls as the jet diverges proximally.

The diameter, D, of the nozzle exit hole 49 is also critical to the use of the device 10 in wound bed preparation. The larger the nozzle diameter, D, the less powerful the fluid jet, the less aggressive the tissue treatment; the smaller the nozzle diameter, D, the more powerful the fluid jet, the more aggressive and sharper the tissue treatment.

For use in wound bed preparation, to perform incremental excision of thin layers (thinner than with the Versajet™ 1 and Versajet™ Plus systems), leave a smooth surface finish on the tissue being debrided (smoother than with the Versajet™ 1 and Versajet™ Plus Systems), and provide ease of user control during debridement (easier control than with the Versajet™ 1 and Versajet™ Plus Systems), with the system parameters of the Versajet™, a deck height, H, in the range of −0.0010″ to 0.0080″ (−0.0254 mm to 0.2032 mm) is necessary, preferably about 0.0005″+0.0050″−0.0010″ (0.0127 mm+0.1270 mm−0.0254 mm), a width, W, in the range of 0.0340″ to 0.0460″ (0.8636 mm to 1.1684 mm) is necessary, preferably about 0.0400″+/−0.0050″ (1.016 mm +/−0.1270 mm), and a nozzle diameter, D, in the range of 0.0042″ to 0.0048″ (0.10668 mm to 0.12192 mm) is necessary, preferably about 0.0045″+/−0.0003″ (0.1143 mm +/−0.00762 mm).

Due to the channel width and deck height combinations, the wound bed preparation device 10 can excise the surface of a wound bed more precisely than the Versajet™ 1 and VersajetTM Plus Systems. With the Versajet™ 1 and Versajet™ Plus Systems the surgeon needs to hold the device slightly off the wound bed surface to avoid cutting too deeply into the tissue, whereas the distal tip 34 of the handpiece 16 of the wound bed preparation device 10 can be placed directly onto the wound bed with a light touch while taking precise layers of the tissue with each stroke of the device. Resting the distal tip 34 of the handpiece 16 on the tissue surface provides the surgeon with more control than trying to hold the device slightly off the wound bed.

A number of embodiments of the invention have been described. Nevertheless, it will be understood that various modifications may be made without departing from the spirit and scope of the invention. Accordingly, other embodiments are within the scope of the following claims.

1.-18. (canceled)

19. A surgical method comprising:
placing a distal tip of a handpiece directly onto a wound bed; and
while maintaining contact between the distal tip and the wound bed, delivering fluid under pressure to the wound bed via a nozzle in the vicinity of a deck formed by distal tip to remove layers of tissue from the wound bed, the nozzle defining a fluid opening and positioned such that a distance between a center of the fluid opening and the deck is in the range of −0.0254 mm to 0.2032 mm.

20. The method of claim 19 wherein delivering fluid to the wound bed further comprises receiving fluid exiting the nozzle through a channel defined by the distal tip and in fluid-flow communication with the nozzle.

21. The method of claim 19 wherein delivering fluid under pressure to the wound bed comprises passing the distal tip across the wound bed in a substantial back and forth motion while maintaining contact between the distal tip and the wound bed.

22. The method of claim 20 wherein delivering fluid to the wound bed comprises passing the distal tip across the wound bed in a substantial back and forth motion while maintaining contact between the distal tip and the wound bed.

23. The method of claim 19 wherein delivering fluid to the wound bed via a nozzle comprises passing fluid through the nozzle having a diameter in the range of 0.10668 mm to 0.12192 mm.

24. The method of claim 20 wherein the fluid exiting the nozzle is received through the channel having a width in the range of 0.8636 mm to 1.1684 mm.
